(19) United States
(12) Patent Application Publication
(10) Pub. No.: US 2024/0226596 A1
(43) Pub. Date: Jul. 11, 2024

(54) PHOTOBIOMODULATION SYSTEMS INCLUDING AN ELECTRODE DISPOSED ON OR OVER A LIGHT EMITTER AND METHODS OF MAKING AND USING

(71) Applicant: Boston Scientific Neuromodulation Corporation, Valencia, CA (US)

(72) Inventors: Rafael Carbunaru, Valley Village, CA (US); Joshua Dale Howard, Sacramento, CA (US); Changfang Zhu, Valencia, CA (US)

(21) Appl. No.: 18/232,621

(22) Filed: Aug. 10, 2023

Related U.S. Application Data
(60) Provisional application No. 63/399,976, filed on Aug. 22, 2022.

(51) Int. Cl. A61N 5/06

(52) U.S. Cl. CPC A61N 5/0601; A61N 5/0622; A61N 2005/063; A61N 2005/0632; A61N 2005/0643; A61N 2005/0652; A61N 2005/0653; A61N 2005/0659; A61N 2005/0661; A61N 2005/0662; A61N 2005/0664

(57) ABSTRACT

An implantable photobiomodulation lead includes a lead body having a proximal end portion and a distal end portion; at least one light emitter disposed along, or coupled to, the distal end portion of the lead body and configured to emit light out of the photobiomodulation lead; and at least one electrode disposed on or over the at least one light emitter and in a path of the light emitted by at least one of the at least one light emitter.

### CROSS-REFERENCE TO RELATED APPLICATIONS

This application claims the benefit under 35 U.S.C. § 119(e) of U.S. Provisional Patent Application Ser. No. 63/399,976, filed Aug. 22, 2022, which is incorporated herein by reference.

### FIELD

The present disclosure is directed to the area of implantable photobiomodulation (PBM) or PBM/electrical stimulation systems and methods of making and using the systems. The present disclosure is also directed to implantable PBM or PBM/electrical stimulation systems that include an electrode disposed on or over a light emitter.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence. Functional electrical stimulation systems have been applied to restore some functionality to paralyzed extremities in spinal cord injury patients. Stimulation of the brain, such as deep brain stimulation, can be used to treat a variety of diseases or disorders.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

Photobiomodulation (PBM) can also provide therapeutic benefits in a variety of diseases and disorders by itself or in combination with electrical stimulation. PBM can provide treatment for symptoms, as well as full or partial relief from pain and other effects of the disease or disorder. A PBM system may include one or more light sources and, often, one or more optical fibers to carry the light to the desired modulation site.

### BRIEF SUMMARY

In one aspect, an implantable photobiomodulation lead includes a lead body having a proximal end portion and a distal end portion; at least one light emitter disposed along, or coupled to, the distal end portion of the lead body and configured to emit light out of the photobiomodulation lead; and at least one electrode disposed on or over the at least one light emitter and in a path of the light emitted by at least one of the at least one light emitter.

In at least some aspects, the at least one electrode is transparent or translucent. In at least some aspects, at least one of the at least one electrode includes graphene, carbon nanotubes, or indium tin oxide.

In at least some aspects, at least one of the at least one electrode is a mesh electrode that allow transmission of at least 50% of light through the mesh electrode. In at least some aspects, at least one of the at least one electrode defines a grid of holes that allow transmission of at least 50% of light through the electrode. In at least some aspects, the least one light emitter includes a plurality of waveguides, wherein at least one of the waveguides extends into or through at least one of the holes of the grid of holes.

In at least some aspects, at least one of the at least one light emitter is a light source. In at least some aspects, at least one of the at least one light emitter is configured to emit light from a distal tip of the photobiomodulation lead. In at least some aspects, at least one of the at least one light emitter is configured to emit light from a side of the photobiomodulation lead. In at least some aspects, at least one of the at least one light emitter is a directional light emitter.

In at least some aspects, the photobiomodulation lead further includes a paddle body attached to the distal end portion of the lead body, wherein at least one of the at least one light emitter is disposed on the paddle body. In at least some aspects, the photobiomodulation lead further includes a cuff body attached to the distal end portion of the lead body, wherein at least one of the at least one light emitter is disposed on the cuff body.

In at least some aspects, the at least one electrode is a plurality of electrodes and the plurality of electrodes includes at least one set of segmented electrodes disposed at a same longitudinal position along the photobiomodulation lead. In at least some aspects, the photobiomodulation lead further includes an optical assembly including at least one of the at least one light emitter and an insulative layer between at least a portion of the at least one electrode and a portion of the optical assembly.

In at least some aspects, at least one of the at least one light emitter is an optical waveguide configured to receive light from a light source and to emit the light out of the photobiomodulation lead. In at least some aspects, at least one of the at least one electrode is disposed on the optical waveguide, the photobiomodulation lead further including a conductive trace extending along the optical waveguide from the at least one of the at least one electrode. In at least some aspects, the photobiomodulation lead further includes the light source disposed within the photobiomodulation lead.

In at least some aspects, the at least one light emitter includes a plurality of light emitters, wherein the plurality of light emitters includes a set of light emitters disposed in a ring within the photobiomodulation lead.

In another aspect, a photobiomodulation system includes any of the photobiomodulation leads of described above and an implantable control module coupled or coupleable to the photobiomodulation lead and configured to control emission of light by the at least one light emitter.

In a further aspect, a method of photobiomodulation of tissue includes emitting light from the at least one light emitter of any of the photobiomodulation leads described above implanted in the tissue.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic side view of one embodiment of a photobiomodulation (PBM) or PBM/electrical stimulation system with a control module and a lead having a light emitter;

FIG. 2 is a schematic side view of a portion of another embodiment of a lead with multiple light emitters;

FIG. 3 is a schematic side view of a control module and a portion of a lead of a yet another embodiment of a photobiomodulation (PBM) or PBM/electrical stimulation system;

FIG. 4 is a schematic side view of one embodiment of a photobiomodulation (PBM) or PBM/electrical stimulation system with a control module and a paddle lead having a light emitter;

FIG. 5 is a schematic exploded perspective view of one embodiment of an optical assembly that attaches to a distal end of a lead;

FIGS. 6A and 6B are schematic perspective views from different angles of one embodiment of an optical assembly with at least one electrode disposed over the optical assembly;

FIG. 7 is a schematic perspective view of another embodiment of an optical assembly with at least one electrode disposed over the optical assembly;

FIG. 8 is a cross-sectional view of a part of the optical assembly of FIGS. 6A and 6B;

FIG. 9 is a schematic top view of one embodiment of a paddle body of a paddle lead with light emitters;

FIG. 10A is a schematic close-up top view of one of the light emitters of the paddle body of FIG. 9;

FIG. 10B is a schematic top view of the light emitter of FIG. 10A with a transparent or translucent electrode disposed over the light emitter;

FIG. 10C is a schematic top view of the light emitter of FIG. 10A with an electrode with a grid of openings disposed over the light emitter;

FIG. 10D is a schematic top view of the light emitter of FIG. 10A with a mesh electrode disposed over the light emitter;

FIG. 11 is a schematic top view of another embodiment of a paddle body of a paddle lead with light emitters illustrating one of one or more light sources and one or more waveguides for transmitting light from the light source(s) to the light emitters;

FIG. 12A is a schematic perspective view of one embodiment of a portion of a cuff lead with light emitters;

FIG. 12B is a schematic view of the interior surface of the cuff body of the cuff lead of FIG. 12A illustrating the light emitters;

FIG. 13A is a schematic side view of one embodiment of a portion of a lead with a light emitter with transparent or translucent electrodes disposed over the light emitter;

FIG. 13B is a schematic side view of one embodiment of a portion of a lead with a light emitter with mesh electrodes disposed over the light emitter;

FIG. 13C is a schematic side view of one embodiment of a portion of a lead with a light emitter with electrodes having a grid of openings disposed over the light emitter;

FIG. 13D is a schematic perspective view of one embodiment of a portion of a lead with a ring of light emitters disposed within the lead;

FIG. 14 is a schematic side view of one embodiment of a portion of a lead with a light emitter, a waveguide extending from the light emitter, and at least one electrode disposed on the waveguide and a conductive wire or trace extending from the at least one electrode;

FIG. 15A is a schematic side view of one embodiment of an optical assembly with side-emitting light emitter(s);

FIG. 15B is a schematic side view of the optical assembly of FIG. 15A with a transparent or translucent electrode disposed over the optical assembly;

FIG. 15C is a schematic side view of the optical assembly of FIG. 15A with a mesh electrode disposed over the optical assembly;

FIG. 15D is a schematic side view of the optical assembly of FIG. 15A with an electrode with a grid of openings disposed over the optical assembly;

FIG. 16A is a schematic top view of one embodiment of an electrode with a grid of openings and optical waveguides extending into or through the openings;

FIG. 16B is a schematic side view of the electrode and optical waveguides of FIG. 16A; and

FIG. 17 is a block diagram of one embodiment of a system for PBM or PBM/electrical stimulation.

### DETAILED DESCRIPTION

The present disclosure is directed to the area of implantable photobiomodulation (PBM) or PBM/electrical stimulation systems and methods of making and using the systems. The present disclosure is also directed to implantable PBM or PBM/electrical stimulation systems that include an electrode disposed on or over a light emitter.

The systems described herein can produce PBM or both PBM and electrical stimulation. In at least some of these embodiments, PBM can be provided through a modification of an electrical stimulation system. PBM may include, but is not necessarily limited to, biomodulation or stimulation resulting from response to particular wavelengths or wavelength ranges of light or from thermal effects generated using light or from any combination thereof.

An implantable PBM or PBM/electrical stimulation system includes at least one light source, such as a light emitting diode (LED), light emitting transistor (LET), laser diode, a vertical cavity side-emitting laser (VCSEL), an organic light emitting diode (OLED), organic light emitting transistor (OLET), a lamp, or any other suitable light source.

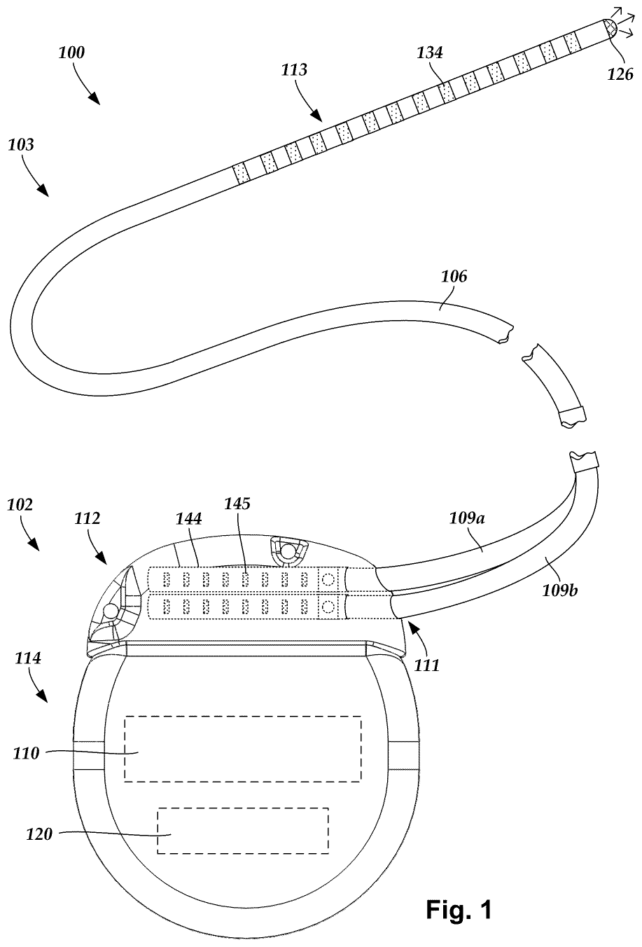

FIG. 1 is a schematic side view of a portion of an embodiment of a PBM or PBM/electrical stimulation system 100 that includes a control module 102 and a lead 103. The control module 102 includes a sealed electronics housing 114 with an electronic subassembly 110 and an optional power source 120. The control module also includes connector housing 112 (which is also often called a header) that houses a control module connector 144 that defines at least one port 111 into which a proximal end 109a, 109b of the lead 103 can be inserted. The control module connector 144 also includes connector contacts 145 disposed within each port 111. The control module 102 (or other device) can define any suitable number of ports including, for example, one, two, three, four, five, six, seven, eight, or more ports. In FIG. 1, the lead 102 is shown coupled into a two ports 111 defined in the control module connector 112. Other embodiments of a control module 102 may have more or fewer components.

When the proximal end 109a, 109b of the lead 102 is inserted into the port 111, the connector contacts 145 can be aligned with a plurality of terminals 132 (FIG. 3) disposed along the proximal end(s) of the lead. Examples of connectors in control modules are found in, for example, U.S. Pat. Nos. 7,244,150 and 8,224,450, which are incorporated herein by reference in their entireties, as well as other references listed herein.

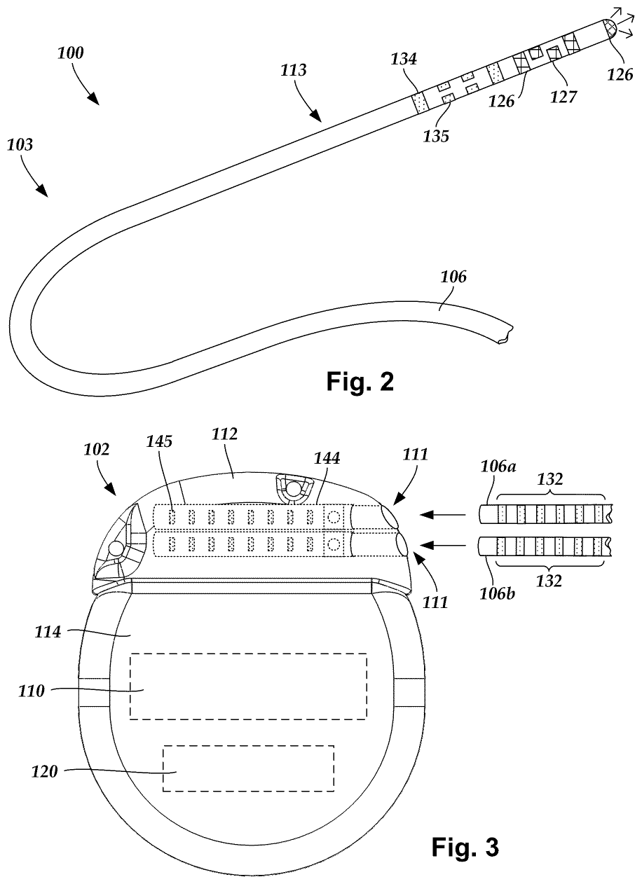

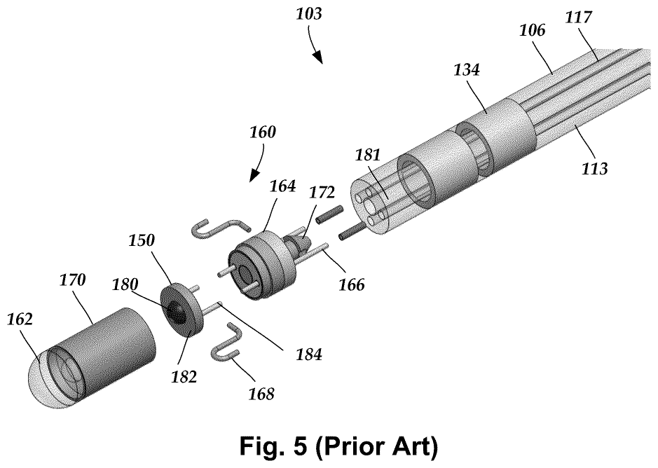

The optional power source 112 can provide power to the electronic subassembly 110. The electronic subassembly 110 is, at least in some embodiments, programmable and is configured to direct the PBM and, if present, electrical stimulation. The electronic subassembly 110 is electrically coupled to the connector contacts 144 and controls a light source 150 (FIG. 5) through signals sent to the connector contacts 144 and through the terminals 168 and conductors 117 (FIG. 5) of the lead 166 to the light source. The electrodes 134 can be ring electrodes, tip electrodes, segmented electrodes 135 (FIG. 2), or any combination thereof.

The lead 103 includes a lead body 106, one or more proximal ends 109a, 109b, one or more distal ends 113, at least one light emitter 126 disposed along the distal end, one or more optional electrodes 134 disposed along the distal end, and one or more optional terminals 132 (FIG. 3) disposed along the proximal end of the lead. Conductors 117 (FIG. 5) extend from the terminals 132 to the optional electrodes 134.

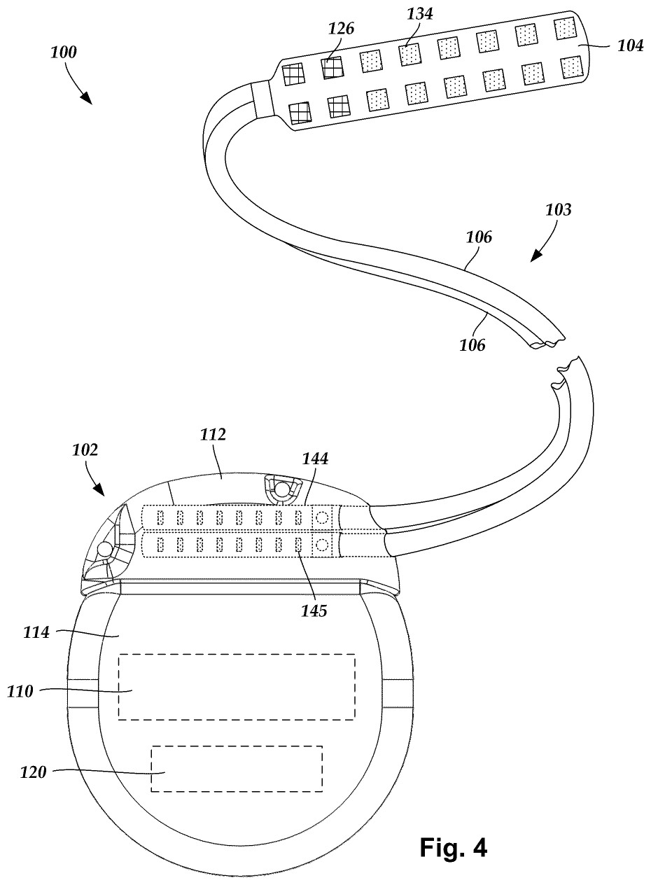

FIG. 4 illustrates a paddle lead 103 which is similar to the lead 103 of FIG. 1 except that the paddle lead 103 includes a paddle body 104 disposed on the distal end of the lead body(ies) 106. The paddle body 104 includes at least one light emitter 126 and optionally includes one or more electrodes 134 distributed on the paddle body. Any arrangement of light emitter(s) and optional electrode(s) can be used including arrangement with one, two, three, four, or more columns and one, two, three, four, or more rows. Any of the rows and columns can be offset from each other.

The light emitter 126 of any of the embodiments described herein (unless indicated otherwise) can be a light source or can be a light emission region of an optical waveguide 136 (FIG. 11) or the like. When the light emitter 126 is a light source, conductors 117 (FIG. 5) extend from the terminals 132 (FIG. 3) to the light emitter to power and operate the light source. Examples of such a light emitter can be found in U.S. Pat. No. 10,335,607, incorporated herein by reference in its entirety.

When the light emitter 126 is a light emission region, the light source can be disposed in the control module 102, lead 103, or other components. Light from the light source is transmitted along one or more optical waveguides 136 (FIG. 11) or the like to the light emission region. Any suitable optical waveguide can be used including, but not limited to, optical fibers, fiber optics, optical plates, lenses, any other suitable light conveyance, or the like or any combination thereof.

Although FIG. 1 illustrates that light emitter 126 emitting light from a tip of the lead 102, it will be understood that a light emitter 126 can emit light from the side of the lead 102, as illustrated in FIG. 2, and may emit light around the entire circumference of the lead or only a portion of the circumference as a segmented light emitter 127, as illustrated in FIG. 2. Although FIG. 1 illustrates a single light emitter 126, it will be understood that a lead 102 can have multiple light emitters (e.g., light sources or light emission regions), as illustrated in FIG. 2, which may be the same or may differ in size, shape, orientation, emission frequency, or the like or any combination thereof.

Examples of PBM and PBM/electrical stimulation systems can be found at, for example, U.S. Pat. Nos. 9,415,154; 10,335,607; and 10,814,140; and U.S. Patent Applications Publications Nos. 2020/0155854; 2021/0008388; 2021/0008389; 2021/0016111; and 2022/0072329, all of which are incorporated herein by reference in their entireties. Examples of electrical and PBM/electrical stimulation systems with leads that can be used or modified to include the elements described herein are found in, for example, U.S. Pat. Nos. 6,181,969; 6,295,944; 6,391,985; 6,516,227; 6,609,029; 6,609,032; 6,741,892; 7,244,150; 7,450,997; 7,672,734; 7,761, 165; 7,783,359; 7,792,590; 7,809,446; 7,949,395; 7,974,706; 8,831,742; 8,688,235; 6,175,710; 6,224,450; 6,271,094; 6,295,944; 6,364,278; and 6,391,985; U.S. Patent Applications Publication Nos. 2007/0150036; 2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; 2011/0005069; 2010/0268298; 2011/0130817; 2011/0130818; 2011/0078900; 2011/0238129; 2011/0313500; 2012/0016378; 2012/0046710; 2012/0165911; 2012/0197375; 2012/0203316; 2012/0203320; and 2012/0203321, all of which are incorporated herein by reference in their entireties.

FIG. 5 illustrates one embodiment of an optical assembly 160 that is coupled to a distal end 113 of lead body 106 of a lead 103 with electrodes 134. Additional details regarding this embodiment of an optical assembly 160 can be found in U.S. Pat. No. 10,335,607, incorporated herein by reference in its entirety. The optical assembly 160 includes the light source 150 (which is a light emitter 126 (FIG. 1)), an emitter cover 162, a feedthrough assembly 164, feedthrough pins 166, conductive cables 168, a metal tube 170, and an anchor 172 to fit in a lumen 181 in the lead 103. The light source 150 emits light in response to electrical signals transmitted along the lead 103. Any suitable light emitter can be used including, but not limited to, a light emitting diode (LED), light emitting transistor (LET), laser diode, organic light emitting diode (OLED), organic light emitting transistor (OLET), or the like. The light source 150 can emit one or more wavelengths of light or can emit light in one or more wavelength bands. The wavelength or wavelength band can be visible, infrared, or ultraviolet light. In at least some embodiments, the light source 150 emits visible light.

In at least some embodiments, the light source 150 includes an emitter element 180, a flange 182 or other housing, and at least two contacts 184. The emitter element 180 may also include a protective material (such as a ceramic or polymeric material) disposed around the actual light emitting structure. The flange 182 can be made of any suitable material and may provide structural or positional stability for the emitter element 180 within the optical assembly 160. The contacts 184 can be pins, pads, or any other structure that provides for electrical coupling of the emitter element 180 to other components, such as the conductive cables 168 or feedthrough pins 166. The conductive cables 168 of the optical assembly 160 can be conductive wires, pins, or any other suitable conductive structure to electrically couple the contacts 184 of the light source 150 to the feedthrough pins 166. It will be understood that in other embodiments, the contacts 184 can be coupled directly to the feedthrough pins 166 without the use of conductive cables.

In at least some embodiments, the emitter cover 162 is disposed over the light source 150 and is, optionally, an optical component such as an optical diffuser or lens. For example, a sapphire optical diffuser dome can be positioned over the light emitter. In other embodiments, the emitter cover 162 may be an optically transparent or translucent material to seal the light source 150 within the optical assembly 160. In some embodiments, the emitter cover 162 can be part of a package that forms the light source 150.

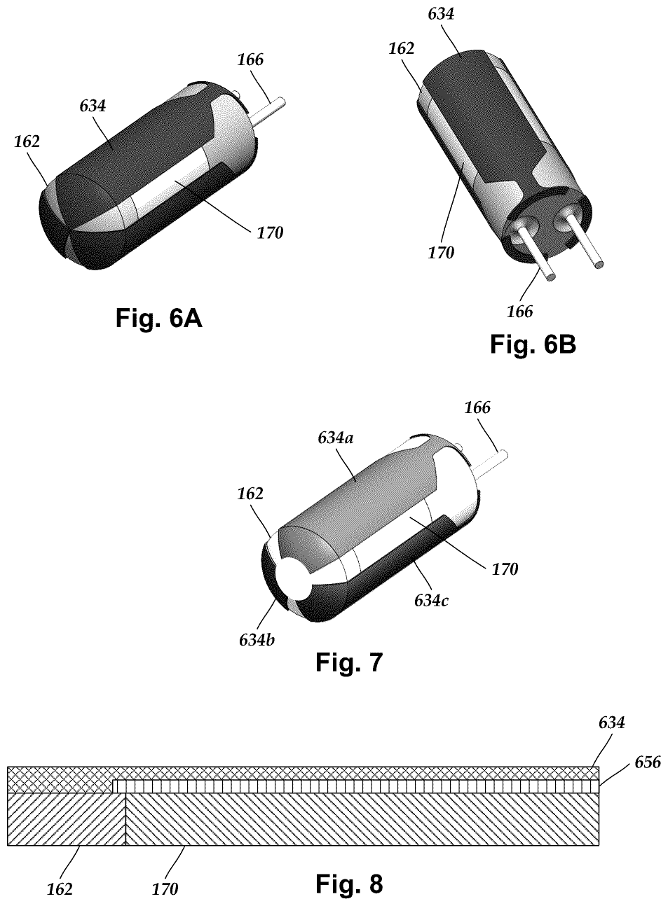

In at least some embodiments, biomodulation and electrical stimulation can be applied to the same, similar, adjacent, nearby, or overlapping target regions. In at least some embodiments, one or more electrodes 634 can be disposed on the optical assembly 160, as illustrated in FIGS. 6A, 6B, and 7. FIGS. 6A and 6B illustrate one embodiment of an optical assembly 160 with an electrode 634 having three different regions disposed thereon. FIG. 7 illustrated one embodiment of an optical assembly 160 with three segmented electrodes 634a, 634b, 634c (for example, directional electrodes) disposed thereon. Other electrode configurations can be used including, but not limited to, one or more ring electrodes, an electrode that covers the entire tip or emitter cover 162 (and, optionally, more) of the optical assembly 160, any suitable number of segmented electrodes (e.g., one, two, three, four, or more segmented electrodes), or the like or any combination thereof.

In at least some embodiments, particularly when a portion of the electrode(s) 634 covers the emitter cover 162 as illustrated in FIGS. 6A, 6B, and 7, the electrode(s) can be formed of a transparent or translucent material, such as graphene, indium tin oxide (ITO), carbon nanotubes, or the like. Alternatively or additionally, in at least some embodiments, the electrode(s) 634 can be formed as a mesh (for example, a mesh 652 of nanowires such as that illustrated in FIG. 10D but differing in the shape of the electrode) or with a grid of openings (such as the grid of openings 654 illustrated in FIG. 10C but differing in the shape of the electrode) through the electrode. In at least some embodiments of any of the electrodes 634 disclosed herein, the electrode 634 allows transmission of at least 50%, 60%, 67%, 70%, 75%, 80%, 90%, or 95% of light from the light emitter, which is directed through the electrode 634. In at least some embodiments, an optical assembly can include a mirror or other reflector (not shown) to reflect light back toward the electrode 634 to increase efficiency. In at least some embodiments, an optical assembly or waveguide can include one or more of the following: refractive index matching materials (such as lenses, adhesives, or the like) or index matching coatings, films, or layers for anti-refection or for increasing transmission through transparent or translucent components.

In at least some embodiments, as illustrated in the cross-section of FIG. 8, an insulating layer 656 of an electrically insulating material, such as parylene C or any other suitable material, is positioned between the electrode(s) 634 and one or more portions (for example, but not limited to, metal or conductive portions) of the optical assembly 160, such as the metal tube 170. The insulating layer 656 and electrode(s) 634 can be disposed on the optical assembly 160 using any suitable methods including, but not limited to, coating, painting, vapor deposition, sputtering, printing, or the like or any combination thereof. The electrode(s) 634 and insulating layer 656 can be patterned using any suitable method including, but not limited to, photolithography, printing, masking, or the like or any combination thereof.

The electrode(s) 634 can be used for any suitable purpose. For example, for cortical stimulation, the electrode(s) can be used for sensing depth and the optical assembly can be used for photobiomodulation or optical stimulation of a larger area of the brain. This may be used to treat conditions, such as Alzheimer's or Parkinson's disease. As another example, for spinal cord stimulation, the electrode(s) 634 can be used to identify a target for photobiomodulation. In at least some embodiments, electrical stim is turned off while photobiomodulation is on. As another example, the electrode(s) 634 can be used for sensing or for providing electrical stimulation or for any combination of the purposes disclosed herein.

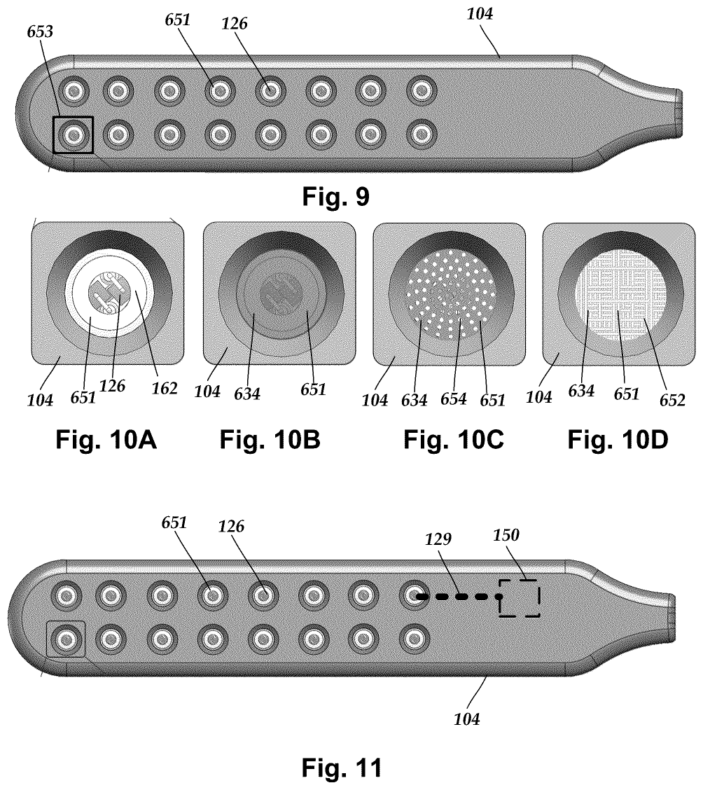

FIG. 9 illustrates one embodiment of a paddle body 104 with openings 651, that expose light emitters 126. FIGS. 10A to 10D illustrate a region 653 of the paddle body 104 around one of the openings 651. In at least some embodiments, as illustrated in FIG. 10A, each of the light emitters 126 includes a light source 150 which is covered by an emitter cover 162 (for example, a lens which may be made of sapphire or any other suitable biocompatible material). In at least some embodiments, one or more light sources 150 can be disposed elsewhere in the paddle body 104, lead 103 (FIG. 4), control module 102 (FIG. 4), or any other suitable location. FIG. 11 illustrates one embodiment of a paddle body 104 in which one or more light sources 150 provide light to one or more light emitters 126 through a waveguide 129 as exemplified for a single light source 150 and a single waveguide 129 in FIG. 11 (any additional light sources and waveguides are not illustrated for clarity of the illustration.)

One or more (or all) of the light emitters 126 are covered by an electrode 634, as illustrated in FIGS. 10B to 10D. In at least some embodiments, the electrode(s) 634 can be formed of a transparent or translucent material, such as graphene, indium tin oxide (ITO), or carbon nanotubes, as illustrated in FIG. 10B. Alternatively or additionally, in at least some embodiments, the electrode(s) 634 can be formed with a grid of openings 654 through the electrode as illustrated in FIG. 10C or as a mesh 652 of nanowires as illustrated in FIG. 10D. The mesh 652 of nanowires can be formed using any suitable method including, but not limited to, deposition, coating, printing, or the like. The grid of openings 654 can be formed in the electrode 634 using any suitable technique including, but not limited to, photolithography, masking, or the like or any suitable method.

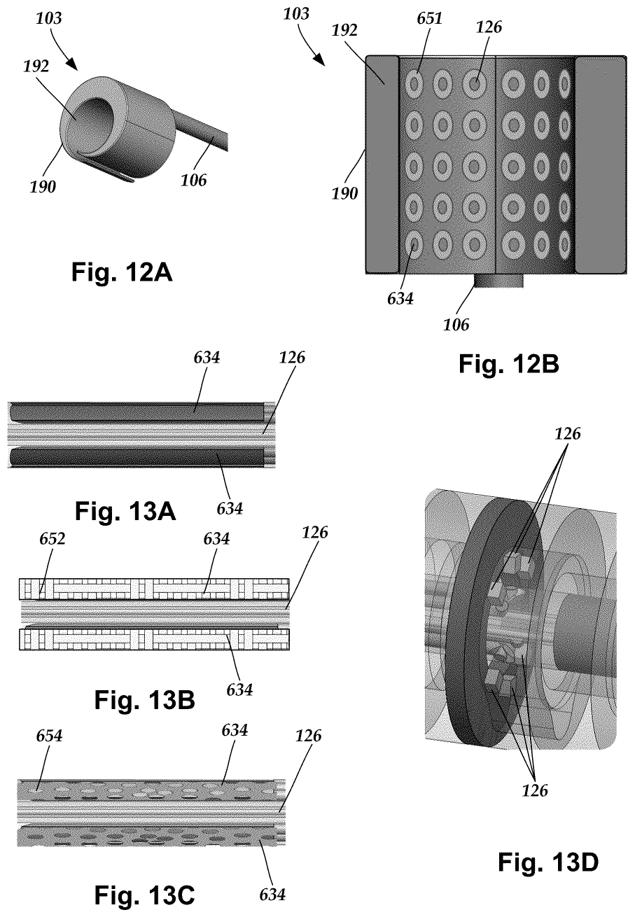

FIGS. 12A and 12B illustrate a portion of one embodiment of a cuff lead 103 that is similar to the paddle lead of FIG. 4 except that the paddle body is replaced by a cuff body 190 attached to the lead body 106. FIG. 12B illustrates the interior surface 192 of the cuff body 190 with openings 651 that expose light emitters 126. One or more of the light emitters 126 are covered by an electrode 634. Any of the arrangements described above for the light emitters 126 and electrodes 634 discussed above with respect to FIGS. 9 to 11 for the paddle body 104 can be used for the cuff body 190. In at least some embodiments, cuff leads 103 are used for peripheral nerve stimulation and photobiomodulation, as well as any other suitable use for the cuff leads.

FIGS. 13A to 13D illustrate additional embodiments of a lead 103 with a light emitter 126 that emits light through a side of the lead (instead or, or in addition to, the tip of the lead—see, FIG. 2) with a portion of one or more electrodes 634 disposed over the light emitter. In at least some embodiments, the light emitter 126 emits light in a ring or one or more portions of a ring around the circumference of the lead 103. In at least some embodiments, multiple light emitters 126 can be positioned at the same longitudinal position along the lead 103 to provide a ring light emitter or to provide multiple directional light emitters 127 (FIG. 2), as illustrated in FIG. 13D.

One or more of the electrodes 634 are disposed over the light emitter(s) 126, as illustrated in FIGS. 13A to 13C. In at least some embodiments, the electrode(s) 634 can be formed of a transparent or translucent material, such as graphene, indium tin oxide (ITO), or carbon nanotubes, as illustrated in FIG. 13A. Alternatively or additionally, in at least some embodiments, the electrode(s) 634 can be formed as a mesh 652 of nanowires as illustrated in FIG. 13B or with a grid of openings 654 through the electrode as illustrated in FIG. 13C.

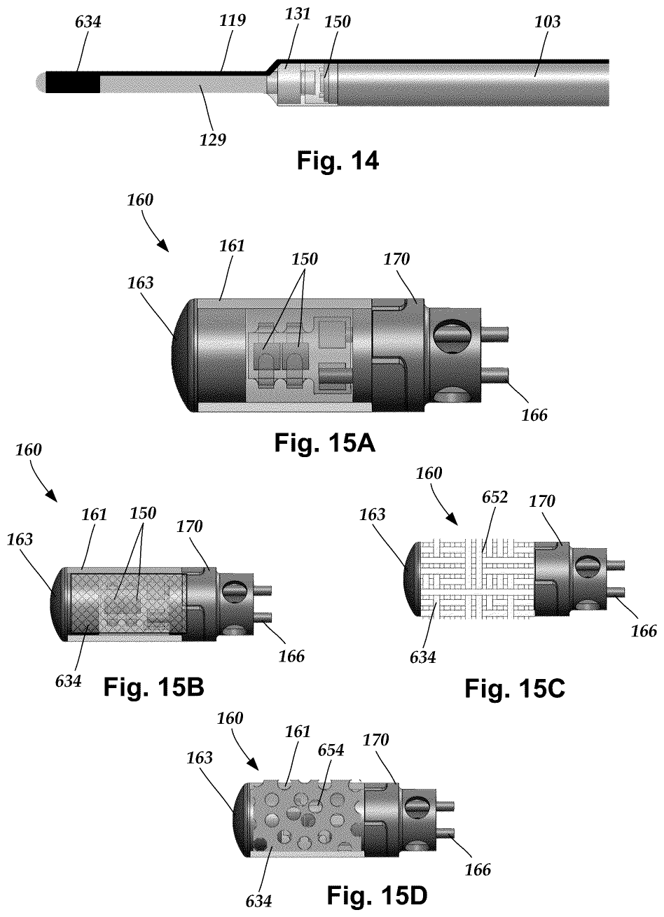

FIG. 14 illustrates a portion of a lead 103 with a light source 150 and an optical waveguide 129 that receives light from the light source (using optics 131). One or more electrodes 634 are disposed on the waveguide 129 with a conductive wire or conductive trace 119 connected to each electrode and disposed along the waveguide, over the light source 150, and on, or within, the lead 103 and coupled to conductors 117 (FIG. 5) within the lead or to terminals 132 (FIG. 3) of the lead. In at least some embodiments, the electrode(s) 634 can be formed of a transparent or translucent material, such as graphene, indium tin oxide (ITO), or carbon nanotubes. Alternatively or additionally, in at least some embodiments, the electrode(s) 634 can be formed as a mesh (such as mesh 652 of FIG. 13B but differing in electrode shape) or with a grid of openings (such as the grid of openings 654 in FIG. 13C but differing in electrode shape) through the electrode. As disclosed above with respect to FIG. 8, an insulative layer 656 can be used to separate the conductive wire or conductive trace 119 or electrode(s) 634 from any conductive components of the light source 150, lead 103, or waveguide 129. In at least some embodiments, a jacket (not shown) is disposed over the conductive wire or conductive trace 119 and waveguide 129 to protect these components from the implant environment. In at least some environments, the jacket may be made of a transparent or translucent material.

FIGS. 15A to 15D illustrate another embodiment of an optical assembly 160 with components that can be the same as those of the embodiment illustrated in FIG. 5 except as described herein. In the optical assembly of FIGS. 15A to 15D, one or more light sources 150 are directed to emit light out of a side of the optical assembly 160, instead of the tip, and there is a transparent or translucent emitter cylinder 161 through which the light is emitted, instead of an emitter cover (see, emitter cover 162 of FIG. 5) as illustrated in FIG. 15A. Replacing the emitter cover 162 of FIG. 5 is a cap 163.

One or more electrodes 634 can be disposed over the optical assembly 160, as illustrated in FIGS. 15B to 15D. In at least some embodiments, particularly when a portion of the electrode(s) 634 covers the emitter cylinder 161, the electrode(s) can be formed of a transparent or translucent material, such as graphene, indium tin oxide (ITO), or carbon nanotubes, as illustrated in FIG. 15B. Alternatively or additionally, in at least some embodiments, the electrode(s) 634 can be formed as a mesh 652, as illustrated in FIG. 15C, or with a grid of openings 654 through the electrode, as illustrated in FIG. 15D. As disclosed above with respect to FIG. 8, an insulative layer 656 can be used to separate the electrode(s) 634 from any conductive components of the optical assembly 160.

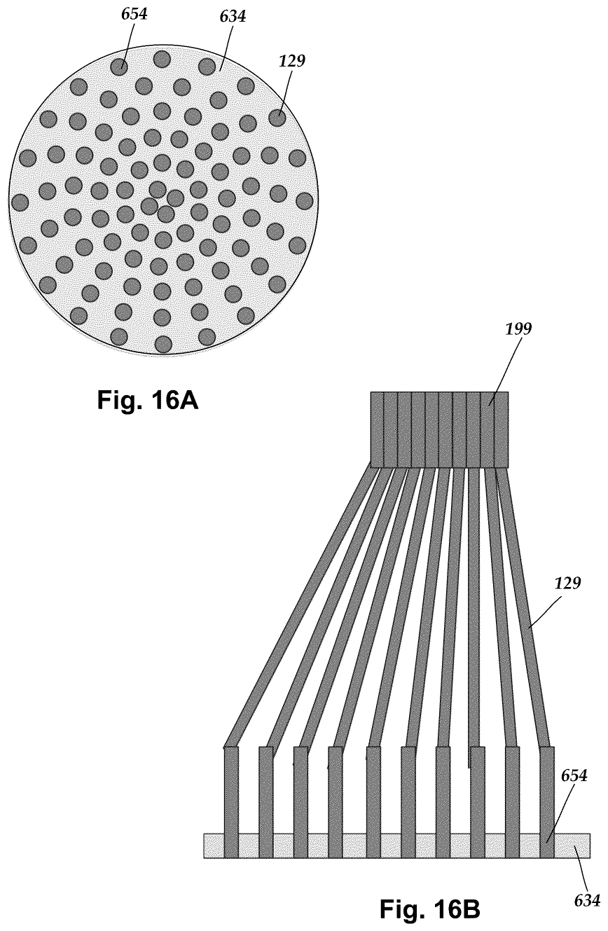

FIGS. 16A and 16B illustrate another arrangement in which waveguides 129 (for example, micro-waveguides) from a bundle 199 or other collection extend into or through an electrode 634 having a grid of openings 654 through the electrode. The bundle 199 of waveguides 129 can receive light from a light source (not shown).

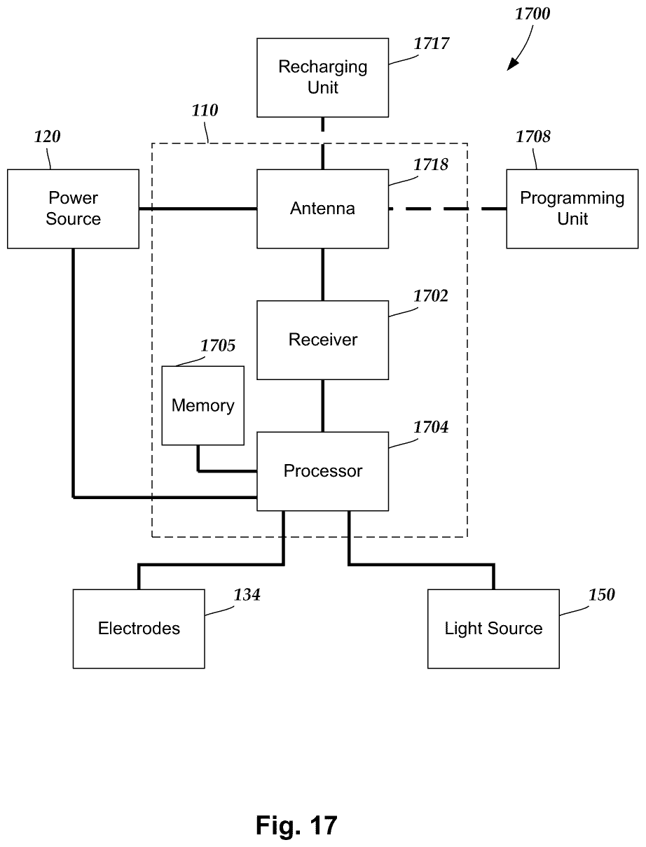

FIG. 17 is a schematic overview of one embodiment of components of an PBM or PBM/electrical stimulation system 1700 including an electronic subassembly 110 disposed within a control module 102 (for example, an implantable pulse generator). It will be understood that the PBM or PBM/electrical stimulation system can include more, fewer, or different components and can have a variety of different configurations including those configurations disclosed in the stimulator references cited herein.

In at least some embodiments, selected components (for example, a power source 170, an antenna 1718, a receiver 1702, a processor 1704, and a memory 1705) of the PBM or PBM/electrical stimulation system can be positioned on one or more circuit boards or similar carriers within a sealed housing of a control module 102. Any suitable processor 1704 can be used and can be as simple as an electronic device that, for example, produces signals to direct or generate PBM or PBM/electrical stimulation at a regular interval or the processor can be capable of receiving and interpreting instructions from an external programming unit 1708 that, for example, allows modification of delivery parameters or characteristics.

The processor 1704 is generally included to control the timing and other characteristics of the PBM or PBM/electrical stimulation system. For example, the processor 1704 can, if desired, control one or more of the timing, pulse frequency, amplitude, and duration of the PBM or PBM/electrical stimulation. In addition, the processor 1704 can select one or more of the electrodes 134 to provide electrical stimulation, if desired. In some embodiments, the processor 1704 selects which of the electrode(s) are cathodes and which electrode(s) are anodes.

Any suitable memory 1705 can be used. The memory 1705 illustrates a type of computer-readable media, namely computer-readable storage media. Computer-readable storage media may include, but is not limited to, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, magnetic storage devices, or any other medium which can be used to store the desired information and which can be accessed by a processor.

The processor 1704 is coupled to a light source 150. Any suitable light source can be used including, but not limited to, LEDs, OLEDs, laser diodes, VCSELs, lamps, light bulbs, or the like or any combination thereof. In at least some embodiments, the PBM or PBM/electrical stimulation system may include multiple light sources. In at least some embodiments, each of the multiple light sources may emit light having a different wavelength or different wavelength range. Any suitable wavelength or wavelength range can be used including, but not limited to, visible, near infrared, and ultraviolet wavelengths or wavelength ranges. A wavelength or wavelength range of a light source may be selected to obtain a specific therapeutic, chemical, or biological effect.

Any power source 170 can be used including, for example, a battery such as a primary battery or a rechargeable battery. Examples of other power sources include super capacitors, nuclear or atomic batteries, fuel cells, mechanical resonators, infrared collectors, flexural powered energy sources, thermally-powered energy sources, bioenergy power sources, bioelectric cells, osmotic pressure pumps, and the like. As another alternative, power can be supplied by an external power source through inductive coupling via an antenna 1718 or a secondary antenna. The external power source can be in a device that is mounted on the skin of the user or in a unit that is provided near the user on a permanent or periodic basis. In at least some embodiments, if the power source 1712 is a rechargeable battery, the battery may be recharged using the antenna 1718 and a recharging unit 1716. In some embodiments, power can be provided to the battery for recharging by inductively coupling the battery to the external recharging unit 1716.

In at least some embodiments, the processor 1704 is coupled to a receiver 1702 which, in turn, is coupled to an antenna 1718. This allows the processor 1704 to receive instructions from an external source, such as programming unit 1708, to, for example, direct the delivery parameters and characteristics. The signals sent to the processor 1704 via the antenna 1718 and the receiver 1702 can be used to modify or otherwise direct the operation of the PBM or PBM/electrical stimulation system. For example, the signals may be used to modify the characteristics or delivery parameters of the PBM or PBM/electrical stimulation system. The signals may also direct the PBM or PBM/electrical stimulation system 1700 to cease operation, to start operation, to start charging the battery, or to stop charging the battery. In other embodiments, the PBM or PBM/electrical stimulation system does not include the antenna 1718 or receiver 1702 and the processor 1704 operates as initially programmed.

In at least some embodiments, the antenna 1718 is capable of receiving signals (e.g., RF signals) from an external programming unit 1708 (such as a clinician programmer or patient remote control or any other device) which can be programmed by a user, a clinician, or other individual. The programming unit 1708 can be any unit that can provide information or instructions to the PBM or PBM/electrical stimulation system 1700. In at least some embodiments, the programming unit 1708 can provide signals or information to the processor 1704 via a wireless or wired connection. One example of a suitable programming unit is a clinician programmer or other computer operated by a clinician or other user to select, set, or program delivery parameters for the PBM or PBM/electrical stimulation system. Another example of the programming unit 1708 is a remote control such as, for example, a device that is worn on the skin of the user or can be carried by the user and can have a form similar to a pager, cellular phone, or remote control, if desired. In at least some embodiments, a remote control used by a patient may have fewer options or capabilities for altering delivery parameters than a clinician programmer.

Optionally, the PBM or PBM/electrical stimulation system 1700 may include a transmitter (not shown) coupled to the processor 1704 and the antenna 1718 for transmitting signals back to the programming unit 1708 or another unit capable of receiving the signals. For example, the PBM or PBM/electrical stimulation system 1700 may transmit signals indicating whether the PBM or PBM/electrical stimulation system 1700 is operating properly or not or indicating when the battery needs to be charged or the level of charge remaining in the battery. The processor 1704 may also be capable of transmitting information about the delivery parameters or characteristics so that a user or clinician can determine or verify the delivery parameters or characteristics.

The above specification provides a description of the manufacture and use of the invention. Since many embodiments of the invention can be made without departing from the spirit and scope of the invention, the invention also resides in the claims hereinafter appended.

What is claimed as new and desired to be protected by Letters Patent of the United States is:

1. An implantable photobiomodulation lead, comprising:
a lead body comprising a proximal end portion and a distal end portion;
at least one light emitter disposed along, or coupled to, the distal end portion of the lead body and configured to emit light out of the photobiomodulation lead; and
at least one electrode disposed on or over the at least one light emitter and in a path of the light emitted by at least one of the at least one light emitter.

2. The photobiomodulation lead of claim 1, wherein the at least one electrode is transparent or translucent.

3. The photobiomodulation lead of claim 2, wherein at least one of the at least one electrode comprises graphene, carbon nanotubes, or indium tin oxide.

4. The photobiomodulation lead of claim 1, wherein at least one of the at least one electrode is a mesh electrode that allow transmission of at least 50% of light through the mesh electrode.

5. The photobiomodulation lead of claim 1, wherein at least one of the at least one electrode defines a grid of holes that allow transmission of at least 50% of light through the electrode.

6. The photobiomodulation lead of claim 5, wherein the least one light emitter comprises a plurality of waveguides, wherein at least one of the waveguides extends into or through at least one of the holes of the grid of holes.

7. The photobiomodulation lead of claim 1, wherein at least one of the at least one light emitter is a light source.

8. The photobiomodulation lead of claim 1, wherein at least one of the at least one light emitter is configured to emit light from a distal tip of the photobiomodulation lead.

9. The photobiomodulation lead of claim 1, wherein at least one of the at least one light emitter is configured to emit light from a side of the photobiomodulation lead.

10. The photobiomodulation lead of claim 1, wherein at least one of the at least one light emitter is a directional light emitter.

11. The photobiomodulation lead of claim 1, further comprising a paddle body attached to the distal end portion of the lead body, wherein at least one of the at least one light emitter is disposed on the paddle body.

12. The photobiomodulation lead of claim 1, further comprising a cuff body attached to the distal end portion of the lead body, wherein at least one of the at least one light emitter is disposed on the cuff body.

13. The photobiomodulation lead of claim 1, wherein the at least one electrode is a plurality of electrodes and the plurality of electrodes comprises at least one set of segmented electrodes disposed at a same longitudinal position along the photobiomodulation lead.

14. The photobiomodulation lead of claim 1, further comprising an optical assembly comprising at least one of the at least one light emitter and an insulative layer between at least a portion of the at least one electrode and a portion of the optical assembly.

15. The photobiomodulation lead of claim 1, wherein at least one of the at least one light emitter is an optical waveguide configured to receive light from a light source and to emit the light out of the photobiomodulation lead.

16. The photobiomodulation lead of claim 15, wherein at least one of the at least one electrode is disposed on the optical waveguide, the photobiomodulation lead further comprising a conductive trace extending along the optical waveguide from the at least one of the at least one electrode.

17. The photobiomodulation lead of claim 15, further comprising the light source disposed within the photobiomodulation lead.

18. The photobiomodulation lead of claim 1, wherein the at least one light emitter comprises a plurality of light emitters, wherein the plurality of light emitters comprises a set of light emitters disposed in a ring within the photobiomodulation lead.

19. A photobiomodulation system, comprising
the photobiomodulation lead of claim 1; and
an implantable control module coupled or coupleable to the photobiomodulation lead and configured to control emission of light by the at least one light emitter.

20. A method of photobiomodulation of tissue, the method comprising:
emitting light from the at least one light emitter of the photobiomodulation lead of claim 1 implanted in the tissue.
